(19) United States
(12) Patent Application Publication
(10) Pub. No.: US 2003/0060462 A1
(43) Pub. Date: Mar. 27, 2003

(54) Therapeutic compounds

(76) Inventors: Robert L. Hoffman, Kalamazoo, MI (US); Michael D. Ennis, Portage, MI (US)

Correspondence Address: FLYNN, THIEL, BOUTELL & TANIS, P.C., 2026 RAMBLING ROAD, KALAMAZOO, MI, 49008-1699, US

(21) Appl. No.: 10/212,664

(22) Filed: Aug. 3, 2002

Related U.S. Application Data
(60) Provisional application No. 60/310,332, filed on Aug. 6, 2001.

(51) Int. Cl. A61K 31/551; C07D 487/22

(52) U.S. Cl. 514/219; 540/555

(57) ABSTRACT

The present invention provides compounds of formula I:

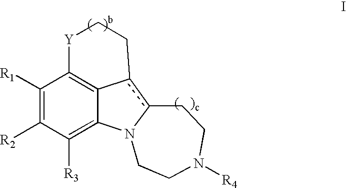

wherein R₁, R₂, R₃, R₄, Y, b, and c have any of the values defined in the specification, as well as pharmaceutical compositions comprising the compounds. The invention also provides therapeutic methods for treating diseases wherein modulation of 5-HT activity is desired, as well as processes and intermediates useful for preparing compounds of formula I.

### CROSS REFERENCE TO RELATED APPLICATIONS

This application claims the benefit of U.S. provisional application Serial No. 60/310,332 filed on Aug. 6, 2001, under 35 USC 119(e)(i), which is incorporated herein by reference in its entirety.

### FIELD OF THE INVENTION

The present invention provides unsubstituted and substituted tetracyclic compounds of formula I as described hereinbelow. These compounds are 5-HT ligands, and are useful for treating diseases wherein modulation of 5-HT activity is desired.

### BACKGROUND OF THE INVENTION

Serotonin has been implicated in a number of diseases and conditions that originate in the central nervous system. These include diseases and conditions related to sleeping, eating, perceiving pain, controlling body temperature, controlling blood pressure, depression, anxiety, schizophrenia, and other bodily states. R. W. Fuller, Biology of Serotonergic Transmission, ed. Neville V. Osborne, John Wiley and Sons (1982), p 221; D. J. Boullin, Serotonin in Mental Abnormalities 1, John Wiley and Sons (1978), p. 316; J. Barchas, et al., Serotonin and Behavior, Academic Press, New York, N.Y. (1973); Barnes, N. M.; A Review Of Central 5-HT Receptors And Their Function, Neuropharmacology, 38, (1999), 1083-1152. Serotonin also plays an important role in peripheral systems, such as the gastrointestinal system, where it has been found to mediate a variety of contractile, secretory, and electrophysiologic effects.

As a result of the broad distribution of serotonin within the body, there is a tremendous interest in drugs that affect serotonergic systems. In particular, receptor-specific agonists and antagonists are of interest for the treatment of a wide range of disorders, including anxiety, depression, hypertension, migraine, obesity, compulsive disorders, schizophrenia, autism, neurodegenerative disorders (for example, Alzheimer's disease, Parkinsonism, and Huntington's chorea), and chemotherapy-induced vomiting. M. D. Gershon, et al., The Peripheral Actions of 5-Hydroxytryptamine, 246 (1989); P. R. Saxena, et al., Journal of Cardiovascular Pharmacology, 15:Supplement 7 (1990).

The major classes of serotonin receptors (5-HT₁₋₇) contain fourteen to eighteen separate receptors that have been formally classified. See Glennon, et al., Neuroscience and Behavioral Reviews, 1990, 14, 35; and D. Hoyer, et al. Pharmacol. Rev. 1994, 46, 157-203. Recently discovered information regarding subtype identity, distribution, structure, and function suggests that it is possible to identify novel, subtype specific agents, having improved therapeutic profiles, for example, fewer side effects.

For example, the 5-HT₂ family of receptors is comprised of 5-HT_{2A}, 5-HT_{2B}, and 5-HT_{2C} subtypes, which have been grouped together on the basis of primary structure, secondary messenger system, and operational profile. All three subtypes are G-protein coupled, activate phospholipase C as a principal transduction mechanism, and contain a seven-transmembrane domain structure. There are distinct differences in the distribution of the three 5-HT₂ subtypes. The 5-HT_{2B} and 5-HT_{2A} receptors are widely distributed in the periphery, while the 5-HT_{2C} receptor has been found only in the central nervous system, being highly expressed in many regions of the human brain. See G. Baxter, et al. Trends in Pharmacol. Sci. 1995, 16, 105-110.

Subtype 5-HT_{2A} has been associated with effects including vasoconstriction, platelet aggregation, and bronchoconstriction, while subtype 5-HT_{2C} has been associated with diseases that include depression, anxiety, obsessive compulsive disorder, panic disorders, phobias, psychiatric syndromes, and obesity. Very little is known about the pharmacologic role of the 5-HT_{2B} receptor. See F. Jenck, et al., Exp. Opin. Invest. Drugs, 1998, 7, 1587-1599; M. Bos, et al., J. Med. Chem., 1997, 40, 2762-2769; J. R. Martin, et al., The Journal of Pharmacology and Experimental Therapeutics, 1998, 286, 913-924; S. M. Bromidge, et al., J. Med. Chem., 1998, 41, 1598-1612; G. A. Kennett, IDrugs, 1998, 1, 4, 456-470; and A. Dekeyne, et al., Neuropharmacology, 1999, 38, 415-423.

U.S. Pat. No. 5,314,900, issued May 24, 1994, discloses compounds having the formula:

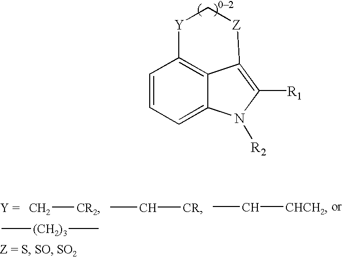

The compounds are reported to act as inhibitors of 5-lipoxygenase and as inhibitors of leukotriene biosynthesis.

There is currently a need for pharmaceutical agents that are useful to treat diseases and conditions associated with 5-HT receptors.

### SUMMARY OF THE INVENTION

In accordance with the present invention, novel compounds which demonstrate useful biological activity, and particularly activity as 5-HT receptor ligands, are provided. Thus, the present invention provides a compound of formula I:

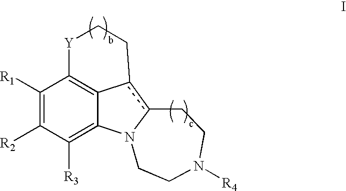

wherein:

b is 1 or 2

c is 0 or 1;

Y is oxy (—O—), thio (—S—), carbonyl (—C(═O)—), NR₅, or CH₂;

the bond represented by ----- is absent or present;

R₁, R₂, and R₃ are each independently hydrogen, halo, —CF₃, —OCF₃, —CN, —NO₂, C₁₋₈alkyl, —OR₆, —SR₆, aryl, (aryl)C₁₋₈alkylene, heteroaryl, (heteroaryl)C₁₋₈alkylene or C₃₋₈ cycloalkyl;

R₄ and R₅ are independently hydrogen, C₁₋₈alkyl, C₃₋₈cycloalkyl, —C(O)O—R₇, —C(O)R₇, —SO₂R₇ or (aryl)C₁₋₈alkylene-; and

each R₆ and R₇ is independently hydrogen, C₁₋₈alkyl, C₃₋₈cycloalkyl, aryl or (aryl)C₁,₈alkylene-;

wherein any aryl or heteroaryl group of R₁, R₂, R₃, R₄, and R₅ is optionally substituted with one or more substituents (e.g. 1, 2, 3, or 4) independently selected from halo, —OH, —CN, —NO₂, —CF₃, —OCF₃, —OR₇, —S(O)₀₋₂C_{1 8}alkyl, C₁₋₈alkyl, C₁₋₈alkoxy, phenyl, C₁₋₈alkanoyl, —C(═O)OR₆, —N(R₆)C(═O)NR₆R₇, —N(R₆)C(═O)OR₆, —NR_{c}R_{d}, —C(═O)NR_{c}R_{d} or —SO₂NR_{c}R_{d};

wherein any C₁₋₈alkyl, or C₁₋₈alkylene group of R₁, R₂, R₃, R₄, and R₅ is optionally substituted with one or more substituents (e.g. 1, 2, 3, or 4) independently selected from oxo (═O), halo, —OH, —CN, —NO₂, —CF₃, —OCF₃, —S(O)₀₋₂C₁₋₈alkyl, C₁₋₈alkyl, C₁₋₈alkoxy, phenyl, C₁₋₈alkanoyl, —C(═O)OR₆, —N(R₆)C(═O) NR₆R₇, —N (R₆)C(═O)OR₆, —NR_{c}R_{d}, —C(═O)NR_{c}R_{d} or —SO₂NR_{c}R_{d};

wherein each R_{c} and R_{d} is independently hydrogen, C₁₋₈alkyl, C₁₋₈alkanoyl, C₁₋₈alkoxycarbonyl, aryl, (aryl)C₁₋₈alkylene-, arylcarbonyl, or aryloxycarbonyl; or R_{c} and R_{d} together with the nitrogen to which they are attached form a pyrrolidino, piperidino, morpholino, or thiomorpholino ring; and

wherein any C₁₋₈alkyl, C₁₋₈alkoxy, C₁₋₈alkanoyl, C₁₋₈alkoxycarbonyl, C₁₋₈alkanoyloxy, C₁₋₈alkylene, or C₃₋₈cycloalkyl, of R₁, R₂, R₃, R₄, R₅, R₆, and R₇ is optionally partially unsaturated;

or a pharmaceutically acceptable salt thereof.

In one embodiment the present invention also provides compounds of formula II:

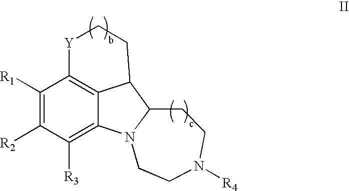

wherein R₁, R₂, R₃, R₄, Y, b, and c have any of the values defined herein.

In another embodiment the present invention also provides a compound of formula III:

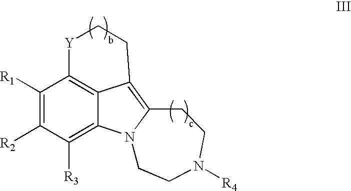

wherein R₁, R₂, R₃, R₄, Y, b, and c have any of the values defined herein.

In another aspect, the present invention also provides:

a pharmaceutical composition comprising a compound of formula I, or a pharmaceutically acceptable salt thereof, and a pharmaceutically acceptable excipient, the composition preferably comprises a therapeutically effective amount of the compound or salt;

a method for treating a disease or condition in a mammal in need thereof, for example, a human, wherein a 5-HT receptor is implicated and modulation of a 5-HT function is desired comprising administering a therapeutically effective amount of a compound of formula I, or a pharmaceutically acceptable salt thereof to the mammal;

a method for treating or preventing a disease or disorder of the central nervous system in a mammal in need thereof comprising administering a therapeutically effective amount of a compound of formula I, or a pharmaceutically acceptable salt thereof to the mammal;

a compound of formula I or a pharmaceutically acceptable salt thereof for use in medical diagnosis or therapy, for example, the treatment or prevention of 5-HT related disease such as anxiety, obesity, depression, or a stress related disease;

the use of a compound of formula I, or a pharmaceutically acceptable salt thereof to prepare a medicament useful for treating or preventing a disease or disorder of the central nervous system in a mammal in need thereof; and

a method for modulating 5-HT receptor function, comprising administering an effective modulatory amount of a compound of formula I, or a pharmaceutically acceptable salt thereof.

The invention also provides novel intermediates and processes disclosed herein that are useful for preparing compounds of formula I.

### DETAILED DESCRIPTION OF THE INVENTION

The compounds of the invention are useful for treating or preventing diseases or disorders of the central nervous system. Specific diseases or disorders of the central nervous system for which a compound of formula I may have activity include, but are not limited to: obesity, depression, schizophrenia, schizophreniform disorder, schizoaffective disorder, delusional disorder, a stress related disease (e.g. general anxiety disorder), panic disorder, a phobia, obsessive compulsive disorder, post-traumatic-stress syndrome, immune system depression, a stress induced problem with the urinary, gastrointestinal or cardiovascular system (e.g., stress incontinence), neurodegenerative disorders, autism, chemotherapy-induced vomiting, hypertension, migraine, headaches, cluster headaches, sexual dysfunction in a mammal (e.g. a human), addictive disorder and withdrawal syndrome, an adjustment disorder, an age-associated learning and mental disorder, anorexia nervosa, apathy, an attention-deficit disorder due to general medical conditions, attention-deficit hyperactivity disorder, behavioral disturbance (including agitation in conditions associated with diminished cognition (e.g., dementia, mental retardation or delirium)), bipolar disorder, bulimia nervosa, chronic fatigue syndrome, conduct disorder, cyclothymic disorder, dysthymic disorder, fibromyalgia and other somatoform disorders, generalized anxiety disorder, an inhalation disorder, an intoxication disorder, movement disorder (e.g., Huntington's disease or Tardive Dyskinesia), oppositional defiant disorder, peripheral neuropathy, post-traumatic stress disorder, premenstrual dysphoric disorder, a psychotic disorder (brief and long duration disorders, psychotic disorder due to medical condition, psychotic disorder NOS), mood disorder (major depressive or bipolar disorder with psychotic features) seasonal affective disorder, a sleep disorder, a specific development disorder, agitation disorder, selective serotonin reuptake inhibition (SSRI) “poop out” syndrome or a Tic disorder (e.g., Tourette's syndrome).

The following definitions are used, unless otherwise described: halo is fluoro, chloro, bromo, or iodo. Alkyl, alkoxy, etc. denote both straight and branched groups; but reference to an individual radical such as “propyl” embraces only the straight chain radical, a branched chain isomer such as “isopropyl” being specifically referred to. Alkylene denotes a divalent straight or branched alkyl group (e.g. methylene (—CH₂—) or ethylene (—CH₂CH₂—)). When alkyl or alkylene can be partially unsaturated, the alkyl chain may comprise one or more (e.g. 1, 2, 3, or 4) double or triple bonds in the chain.

The term “aryl,” alone or in combination, is a phenyl group, or an ortho-fused bicyclic carbocyclic group having nine to ten ring atoms in which at least one ring is aromatic. The term “aryl” is sometimes abbreviated as “Ar.”

The term “heteroaryl” is a radical of a monocyclic aromatic ring containing five or six ring atoms consisting of carbon and 1, 2, 3, or 4 heteroatoms each selected from the group consisting of non-peroxide oxygen, sulfur, and N(Z) wherein Z is absent or is H, C₁₋₄alkyl, phenyl or benzyl, as well as a radical of an ortho-fused bicyclic heterocycle of about eight to ten ring atoms derived therefrom, particularly a benz-derivative or one derived by fusing a propylene, trimethylene, or tetramethylene diradical thereto. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, furazanyl, furyl, imidazolyl, imidizolyl, indazolyl, indolisinyl, indolyl, indolyl, isobenzofuranyl, isoindolyl, isoquinolyl, isothiazolyl, isothiazolyl, isoxazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, xanthenyl, and the like.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, tautomeric, or stereoisomeric form, or mixture thereof, of a compound of the invention, which possesses the useful properties described herein, it being well known in the art how to prepare optically active forms, for example, by resolution of the racemic form by recrystallization techniques, by synthesis from optically-active starting materials, by chiral synthesis, or by chromatographic separation using a chiral stationary phase, and how to determine 5-HT activity using the standard tests which are well known in the art.

The carbon atom content of various hydrocarbon-containing moieties is indicated by a prefix designating the minimum and maximum number of carbon atoms in the moiety, that is, the prefix Cᵢ₋ⱼ indicates a moiety of the integer “i” to the integer “j” carbon atoms, inclusive. Thus, for example, C₁₋₈alkyl refers to alkyl of one to eight carbon atoms, inclusive.

The compounds of the present invention are generally named according to the IUPAC or CAS nomenclature system. Abbreviations which are well known to one of ordinary skill in the art may be used, for example, “Ph” for phenyl, “Me” for methyl, “Et” for ethyl, “h” for hour or hours and “rt” for room temperature.

To the extent that any numerical range is recited in connection with any aspect of the inventive compounds, for example, dosages, treatment regimens, and the like, the range expressly includes all numerals, integer and fractional, falling within the range.

Specific and preferred values listed below for radicals, substituents, and ranges, are for illustration only; they do not exclude other defined values or other values within defined ranges.

Specifically, C₁₋₈ alkyl can be, for example, methyl, ethyl, propyl, isopropyl, butyl, iso-butyl, sec-butyl, pentyl, 3-pentyl, hexyl, heptyl or octyl; C₁₋₈alkoxy and C₁₋₈alkoxy can include, for example, be methoxy, ethoxy, propoxy, isopropoxy, butoxy, iso-butoxy, sec-butoxy, pentoxy, 3-pentoxy, hexyloxy, heptyloxy, and octyloxy; C₃₋₈ cycloalkyl, can be, for example, cyclopropyl, cyclobutyl, cyclopentyl, cyclohexyl, cycloheptyl or cyclooctyl; C₃₋₈cycloalkenyl, can be, for example, cyclopropenyl, cyclobutenyl, cyclopentenyl, cyclohexenyl, cycloheptenyl or cyclooctenyl, with at least one unsaturated carbon-carbon bond within or without the carbocycle; C₁₋₈ haloalkyl, can be, for example, methyl, ethyl, propyl, isopropyl, butyl, iso-butyl, sec-butyl, pentyl, 3-pentyl, hexyl, heptyl or octyl, with one or more halogen atom substituents; C₁₋₈alkanoyl can be acetyl, propanoyl, butanoyl, pentanoyl, 4-methylpentanoyl, hexanoyl, or heptanoyl; C₁₋₈alkoxycarbonyl can be methoxycarbonyl, ethoxycarbonyl, propoxycarbonyl, isopropoxycarbonyl, butoxycarbonyl, pentoxycarbonyl, hexyloxycarbonyl, heptyloxycarbonyl, or octyloxycarbonyl; C₁₋₈alkanoyloxy can be acetyloxy, propanoyloxy, butanoyloxy, isobutanoyloxy, pentanoyloxy, hexanoyloxy, heptanoyloxy, or octanoyloxy; aryl can be, for example, phenyl, indenyl, or naphthyl; and heteroaryl can be, for example, furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl or its N-oxide, thienyl, pyrimidinyl or its N-oxide, indolyl, isoquinolyl or its N-oxide, or quinolyl or its N-oxide.

A specific value for b is 1.

Another specific value for b is 2.

A specific value for c is 0.

Another specific value for c is 1.

A specific value for R₁ is H.

A specific value for R₁ is halo, C₁₋₈alkyl, —OR₆, or —SR₆.

Another specific value for R₁ is C₁₋₈alkyl.

A specific value for R₁ is halo.

Another specific value for R₁ is aryl.

Another specific value for R₁ is substituted aryl.

Another specific value for R₁ is aryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

Another specific value for R₁ is phenyl, optionally substituted with one or more fluoro, chloro, bromo, —OH, —CN, —NO₂, —CH₃, —CF₃, —OCF₃, methoxy, ethoxy, propoxy, or isopropoxy.

Another specific value for R₁ is phenyl, substituted at the 2-position with one or more fluoro, chloro, bromo, —OH, —CN, —NO₂, —CH₃, —CF₃, —OCF₃, methoxy, ethoxy, propoxy, or isopropoxy.

Another specific value for R₁ is 2-ethoxyphenyl, 2-trifluoro-phenyl, 2-chlorophenyl or 2-methylphenyl.

Another specific value for R₁ is heteroaryl.

Another specific value for R₁ is substituted heteroaryl.

Another specific value for R₁ is heteroaryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

Another specific value for R₁ is heteroaryl, optionally substituted with one or more fluoro, chloro, bromo, —OH, —CN, —NO₂, —CF₃, —OCF₃, C₁₋₈alkyl, or C₁₋₈alkoxy.

A specific value for R₂ is H.

A specific value for R₂ is halo, C₁₋₈alkyl, —OR₆, or —SR₆.

Another specific value for R₂ is C₁₋₈alkyl.

A specific value for R₂ is halo.

Another specific value for R₂ is aryl or heteroaryl.

Another specific value for R₂ is substituted aryl.

Another specific value for R₂ is aryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

Another specific value for R₂ is phenyl, optionally substituted with one or more fluoro, chloro, bromo, —OH, —CN, —NO₂, —CH₃, —CF₃, —OCF₃, methoxy, ethoxy, propoxy, or isopropoxy.

Another specific value for R₂ is phenyl, substituted at the 2-position with one or more fluoro, chloro, bromo, —OH, —CN, —NO₂, —CH₃, —CF₃, —OCF₃, methoxy, ethoxy, propoxy, or isopropoxy.

Another specific value for R₂ is 2-ethoxyphenyl, 2-trifluoro-phenyl, 2-chlorophenyl or 2 methylphenyl.

Another specific value for R₂ is heteroaryl.

Another specific value for R₂ is substituted heteroaryl.

Another specific value for R₂ is heteroaryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

A specific value for R₃ is H.

A specific value for R₃ is halo, C₁₋₈alkyl, —OR₆, or —SR₆.

Another specific value for R₃ is C₁₋₈alkyl.

A specific value for R₃ is halo.

Another specific value for R₃ is aryl or heteroaryl.

Another specific value for R₃ is substituted aryl.

A specific value for R₃ is aryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

Another specific value for R₃ is phenyl, optionally substituted with one or more fluoro, chloro, bromo, —OH, —CN, —NO₂, —CH₃, —CF₃, —OCF₃, methoxy, ethoxy, propoxy, or isopropoxy.

Another specific value for R₃ is phenyl, substituted at the 2-position with one or more fluoro, chloro, bromo, —OH, —CN, —CH₃, —NO₂, —CF₃, —OCF₃, methoxy, ethoxy, propoxy, or isopropoxy.

Another specific value for R₃ is 2-ethoxyphenyl, 2-trifluoro-phenyl, 2-chlorophenyl or 2-methylphenyl.

Another specific value for R₃ is heteroaryl.

Another specific value for R₃ is substituted heteroaryl.

Another specific value for R₃ is heteroaryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

A more specific value for R₃ is an alkoxy substituted heteroaryl.

A specific value for R₄ is H.

Another specific value for R₄ is alkyl.

A more specific value for R₄ is C₁₋₈ alkyl.

A more specific value for R₄ is a protecting group.

A more specific value for R₄ is —C(O)O-t-Bu, —C(O)CF₃, —(O)OCH₂Ph, —CH₂—Ph, —SO₂CH₃ or —SO₂Ph.

A more specific value for R₄ is —C(O)O-t-Bu, —C(O)CF₃, —(O)OCH₂Ph, or —CH₂—Ph.

A specific group of compounds are compounds of formula I, II, or III, wherein b=1, c=0; or a pharmaceutically acceptable salt thereof.

A specific group of compounds are compounds of formula I, II, or III, wherein b=1, c=1; or a pharmaceutically acceptable salt thereof.

A specific group of compounds are compounds of formula I, II, or III, wherein b=2, c=0; or a pharmaceutically acceptable salt thereof.

A specific group of compounds are compounds of formula I, II, or III, wherein b=2, c=1; or a pharmaceutically acceptable salt thereof.

A specific group of compounds are compounds of formula I, II, or III, wherein Y is —O—; or a pharmaceutically acceptable salt thereof.

A specific group of compounds are compounds of formula I, II, or III, wherein Y is —S—; or a pharmaceutically acceptable salt thereof.

A specific group of compounds are compounds of formula I, II, or III, wherein Y is —NH—; or a pharmaceutically acceptable salt thereof.

A specific group of compounds are compounds of formula I, II, or III, wherein Y is —NR₅—; or a pharmaceutically acceptable salt thereof.

A specific group of compounds are compounds of formula I, II, or III, wherein Y is —CH₂—; or a pharmaceutically acceptable salt thereof.

A specific group of compounds are compounds of formula I, II, or III, wherein Y is —C(═O)—; or a pharmaceutically acceptable salt thereof.

A specific group of compounds are of formula I, II, or III, wherein R₁ is hydrogen and R₃ is aryl, optionally substituted with one or more, such as 1, 2, 3, or 4, substituents such as halo, —OH, —CN, —NO₂, —CF₃, —OCF₃, —S(O)₀₋₂C₁₋₈alkyl, C₁₋₈alkyl, C₁₋₈alkoxy, phenyl, C₁₋₈alkanoyl, NR_{c}R_{d}, or —C(═O)NR_{c}R_{d}, —SO₂NR_{c}R_{d}.

Another specific group of compounds are of formula I, II, or III, wherein R₃ is hydrogen and R₁ is aryl, optionally substituted with one or more, such as halo, —OH, —CN, —NO₂, —OR₇, —CF₃, —OCF₃, —S(O)₀₋₂C₁₋₈ alkyl, C₁₋₈alkyl, phenyl, C₁₋₈alkanoyl, NR_{c}R_{d}, or —C(═O)NR_{c}R_{d}, —SO₂NR_{c}R_{d}, -halo, -hydroxy, -cyano, -nitro, -trifluoromethyl, -trifluoromethoxy, —S(O)₀₋₂C₁₋₈ alkyl, C₁₋₈alkyl, C₁₋₈alkoxy, phenyl, C₁₋₈alkanoyl, NR_{c}R_{d}, —C(═O)NR_{c}R_{d}, or —SO₂NR_{c}R_{d}.

Another specific group of compounds are of formula I, II, or III, wherein b is 1; and c is 1; and Y is oxy.

Another specific group of compounds are of formula I, II, or III, wherein b is 1; and c is 0; and Y is oxy.

Another specific group of compounds are of formula I, II, or III, wherein b is 2; and c is 1; and Y is oxy.

Another specific group of compounds are of formula I, II, or III, wherein b is 2; and c is 0; and Y is oxy.

Another specific group of compounds are of formula I, II, or III, wherein b is 1; and c is 1; and Y is thio.

Another specific group of compounds are of formula I, II, or III, wherein b is 1; and c is 0; and Y is thio.

Another specific group of compounds are of formula I, II, or III, wherein b is 2; and c is 1; and Y is thio.

Another specific group of compounds are of formula I, II, or III, wherein b is 2; and c is 0; and Y is thio.

Another specific group of compounds are of formula I, II, or III, wherein b is 1; and c is 1; and Y is carbonyl.

Another specific group of compounds are of formula I, II, or III, wherein b is 1; and c is 0 and Y is carbonyl.

Another specific group of compounds are of formula I, II, or III, wherein b is 2; and c is 1; and Y is carbonyl.

Another specific group of compounds are of formula I, II, or III, wherein b is 2; and c is 0 and Y is carbonyl.

Another specific group of compounds are of formula I, II, or III, wherein b is 1; and c is 1; and Y is —N(R₅)—.

Another specific group of compounds are of formula I, II, or III, wherein b is 1; and c is 0; and Y is —N(R₅)—.

Another specific group of compounds are of formula I, II, or III, wherein b is 2; and c is 1; and Y is —N(R₅)—

Another specific group of compounds are of formula I, II, or III, wherein b is 2; and c is 0; and Y is —N(R₅)—.

Another specific group of compounds are of formula I, II, or III, wherein b is 1; c is 1; and Y is —CH₂—.

Another specific group of compounds are of formula I, II, or III, wherein b is 1; and c is 0; and Y is —CH₂—.

Another specific group of compounds are of formula I, II, or III, wherein b is 2; and c is 1; and Y is —CH₂—.

Another specific group of compounds are of formula I, II, or III, wherein b is 2; and c is 0; and Y is —CH₂—.

Specifically, the invention also provides a method for treating or preventing anxiety, obesity, depression, schizophrenia, a stress related disease such as a general anxiety disorder, panic disorder, a phobia, obsessive compulsive disorder, post-traumatic-stress syndrome, immune system depression, a stress induced problem with the gastrointestinal or cardiovascular system, or sexual dysfunction in a mammal, for example a human, comprising administering a therapeutically effective amount of a compound of formula I, or a pharmaceutically acceptable salt thereof to the mammal.

Specifically, the invention also provides a method of treating or preventing anxiety, obesity, depression, or a stress related disease, comprising administering to a mammal, for example a human, in need of such treatment, a therapeutically effective amount of a compound of formula I or a pharmaceutically acceptable salt thereof.

Specifically, the invention also provides the use of a compound of formula I or a pharmaceutically acceptable salt thereof to prepare a medicament for treating or preventing anxiety, obesity, depression, schizophrenia, a stress related disease, such as a general anxiety disorder, panic disorder, a phobia, obsessive compulsive disorder, post-traumatic-stress syndrome, immune system depression, a stress induced problem with the gastrointestinal or cardiovascular system, or sexual dysfunction in a mammal, for example a human.

Specifically, the invention also provides the use of a compound of formula I or a pharmaceutically acceptable salt thereof to prepare a medicament for treating or preventing anxiety, obesity, depression, or a stress related disease in a mammal, for example a human.

The invention also provides processes and intermediates useful for preparing compounds of formula I. For example, an intermediate useful for preparing a compound of formula I which is a corresponding compound of formula I wherein R₄ is a suitable protecting group. Thus, the invention provides a compound of formula I wherein R₄ is a suitable amine protecting group, and wherein R₁, R₂, R₃, Y, b, and c have any of the values, specific values or preferred values defined herein. Suitable amine protecting groups, as well as methods for their preparation and removal are well known in the art, for example, see Greene, T. W.; Wutz, P. G. M. “Protecting Groups In Organic Synthesis” third edition, 1999, New York, John Wiley & sons, Inc. Preferred protecting groups include benzyloxycarbonyl (CBZ) and benzoyl.

The invention also provides novel intermediate compounds that are useful in preparing compounds of formula I, for example, the formulas A9, B4, C5, C7, D6, D8, E4, E7, F3, F4, G3, H1, H4, H5, I1, and I2, as shown in Charts C to I below.

The invention also provides a method for preparing a compound of formula II comprising reducing the corresponding compound of formula III, for example, as indicated in Charts C, D, E, and F.

The invention also provides a method for preparing a compound of formula I wherein R₄ is hydrogen, comprising deprotecting a corresponding compound of formula I wherein R₄ is a suitable nitrogen protecting group, see for example, Chart H, step 3; and

The invention also provides a method for preparing a compound of formula I wherein R₄ is other than hydrogen, comprising alkylating or acylating a corresponding compound of formula I wherein R₄ is a suitable C₁₋₈ alkyl, C₃₋₈ cycloalkyl or (aryl) C₁₋₈ alkylene group, see for example, Chart I, step 2.

The invention also provides intermediate salts that are useful for preparing or purifying compounds of formula I. Suitable methods for preparing salts are known in the art and are disclosed herein. As will be apparent to one skilled in the art, such salts can be converted to the corresponding free-base or to another salt using known methods.

Compounds of the invention can generally be prepared using the synthetic schemes illustrated in the Charts A to I below. Starting materials can be prepared by procedures described in these schemes or by procedures that would be well known to one of ordinary skill in organic chemistry. The variables used in the Schemes are as defined below or as in the claims.

The following describes the preparation of compounds of the present invention. All of the starting materials are prepared by procedures described herein or by procedures that would be well known to one of ordinary skill in organic chemistry.

Compounds of formula I where R₁, R₂, R₃, and R₄ are as defined above can be prepared from azepinoindole tricyclic structure A9, where Z can be, for example, but is not limited to I, Br, thioalkyl of the formula —SR, alkoxy of the formula —OR, alkylamines of the formula —NHR or —NRR. The production of azepinoindole derivatives A9 is depicted in chart A starting from substituted ortho-halonitrobenzene derivatives (for indoles from ortho-halonitrobenzenes derivatives see Sundberg, R. J.; Indoles; Academic Press; 1996; 20-24; Academic Press, Inc.; New York). In addition to commercial sources, the preparation of substituted ortho-halonitrobenzene derivatives A1 is well documented in the chemical literature with many well established methodologies described. For example, see Liedholm, B.; Acta Chem. Scand.; 47; 7; 1993; 701-705 (where Z=Br and X=I ) or Kaszynski, P.; Tetrahedron; 56; 2; 2000; 165-174 (where Z=SR7 and X=Cl) or see Krolski, M. E.; J. Org. Chem.; 53; 6; 1988; 1170-76 and Cereghetti, M.; Tet. Lett.; 37; 30; 1996; 5343-46 (where Z=OR7 and X=Br or I). Palladium catalyzed Sonogashira coupling of a substituted ortho-halonitrobenzene derivative with 3-butyn-1-ol provides A2 (for examples of aryl chlorides in the Sonogashira coupling see Fu, G. C.; Org. Lett; 2; 12; 2000; 1729-31). Reduction of the nitro moiety by SnCl₂ in an alcoholic solvent or by catalytic hydrogenation in the presence of a transition metal such as palladium, rhodium or nickel (see Hudlicky, M. Reductions in Organic Chemistry, 1984, Ellis Horwood, Ltd., Chichester, UK) furnishes the corresponding anilines A3. Alkylation with ethylbromo acetate in a polar solvent such as dimethylformamide (DMF) and with a base such as diisopropylethylamine (DIPEA) or an inorganic base such as sodium phosphate affords A4 derivatives (Tisnes, P.; Tetrahedron; 48; 21; 1992; 4347-58). Cyclization of A4 by transition metal catalysis with CuI (Kuyper, L. F.; J. Med. Chem.; 39; 1996; 892-903) or a Pd(II) species (Stille, J. K.; J. Org. Chem.; 54; 1989; 5856) in a solvent such as dimethylsulfoxide (DMSO), DMF or acetonitrile (CH₃CN) at elevated temperatures produces indole derivatives A5. Conversion of the terminal alcohol moiety of A5 to a suitable leaving group such as mesylate or halogen followed by nucleophilic displacement with an azide source (for a recent procedure employing trimethylsilylazide (TMSN₃) and tetrabutylammonium fluoride (TBAF) see DeShong, P.; J. Org. Chem.; 64; 1999; 3171-77) in a solvent such as CH₃CN provides A6. Treatment of the azide with triphenylphosphine (Ganem, B.; J. Org. Chem.; 52; 22; 1987; 5044-46) in a solvent such as water and THF or an alcoholic solvent affords the corresponding amine. Heating in an alcoholic solvent induces intramolecular cyclization to give lactam derivatives A7. Reduction of A7 by the action of a reducing agent such as lithiumaluminum hydride (LAH) or diisobutylaluminum hydride (DIBAL) in dichloromethane or an ether solvent provides the corresponding amine derivatives A8 (for additional reduction procedures see Larock, R. C., Comprehensive Organic Transformations, 1989, VCH Publishers, New York). Protection of the azepino nitrogen with various protecting groups such as the tert-butylcarbamate under conditions described in Protective Groups in Organic Synthesis, 2^{nd} Edition (Greene and Wuts; 1991; John Wiley and Sons, Inc.; New York) furnishes A9 derivatives. For an alternative approach for the preparation of A9 derivatives see the above mentioned commonly owned and assigned U.S. Ser. No. 09/803,242, “2,3,4,5-Tetrahydro-lH-[1,4]Diazepino[1,7-a]Indole Compounds”.

Compounds of Formula I can be prepared from, for example, tetrahydropyrazinoindole tricyclic structures B4 where Z can be but is not limited to I, Br, thioalkyl of the formula —SR, alkoxy of the formula —OR, alkylamines of the formula —NHR or —NRR. The production of tetrahydropyrazinoindole derivatives B4 is depicted in chart B starting from substituted indole-2-carboxylate derivatives (for examples of B4 derivatives by this route see Sharanabasava, R.; Indian J. Chem. Sect. B.; 28B; 12; 1989; 1065-8). In addition to commercial sources, the preparation of substituted indole-2-carboxylate derivatives B1 are well documented in the chemical literature with many well established methodologies for their preparation being described. For example, see Moody, C. J.; J. Chem. Soc. Perkin. Trans. 1; 1986; 483 (where Z=SR_{c}) or Fagan, G. P.; J. Med. Chem.; 31; 5; 1988; 944-48 (where Z=Br) or Ghosh, S. C.; Chem. Comm.; 11; 2000; 979-80 (where Z=OR_{c}). For a general approach to indole-2-carboxylate derivatives B1 by condensation of an aromatic aldehyde with an azidoacetate enolate and subsequent thermolysis of the resulting α-azidocinnamate see Indoles; Sundberg, R. J.; 1996; 44-47 and Moody, C. J.; J. Chem. Soc. Perkin.Trans.1; 1984; 1333. Alkylation of an indole-2-carboxylate derivative B1 with chloroacetonitrile in the presence of a base such as sodium hydride and polar aprotic solvent such as DMF affords B2. Reaction of B2 with LAH in an ether solvent provides reductive cyclization derivatives B3. Protection of the piprazino nitrogen as the tert-butylcarbamate or trifluoromethylamide affords B4 derivatives.

Chart C illustrates the production of compounds of Formula. I. Reaction of C1, where X can be but is not limited to I, Br, Cl, OR7, and triflate according to Chart A and B, with oxalyl chloride in an appropriate solvent such as diethyl ether or dichloromethane at reduced temperatures affords an intermediate α-keto acid chloride. Alcoholysis with ethanol or methanol furnishes the corresponding α-keto ester (Humber, L. G; J.Med.Chem.; 31; 9; 1988; 1712-1719). Removal of the carbamate protecting group provides C2. Reduction of the α-ketoester in a single step with LAH in an ether solvent such as THF or diethylether at elevated temperatures affords the corresponding primary alcohol (Kita, Y.; Synthesis; 5; 1999; 885-897). Alternatively, a two step procedure by initial reduction with lithium aluminum hydride in an appropriate solvent such as tetrahydrofuran produces the corresponding diol (Bosch, J.; J. Org. Chem.; 58; 1993; 7756-67). Further reduction of the secondary alcohol by the action of triethyl silane in the presence of trifluoroacetic acid (TFA) and an appropriate solvent such as dichloromethane provides the corresponding primary alcohol (Rapoport, H.; Synthesis; 9; 1986; 735-37). Protection of the azepino nitrogen with di-tert-butyl dicarbonate provides C3. Conversion of the terminal alcohol moiety of C3 to a suitable leaving group such as mesylate followed by nucleophilic displacement with an amine derivative such as benzyl amine in a polar solvent such as CH₃CN or DMF furnishes C4 derivatives wherein R7 is, for example, benzyl. Reaction of C4 with a palladium catalyst such as Pd(PPh₃)₄ in the presence of a base such as sodium tert-butoxide and/or potassium carbonate in a high boiling solvent such as toluene at elevated temperatures furnishes C5 (Buchwald, S. L.; J. Am. Chem. Soc.; 118; 5; 1028-1030). Deprotection of the tert-butylcarbamate with TFA in a solvent such as dichloromethane provides C6. Reduction of indole derivatives C6 by a reducing agent such as sodium cyanoborohydride in the presence of an acid such as TFA or acetic acid (see Indoles; Sundberg, R. J.; 1996) affords the corresponding indoline derivatives C7. Removal of the N-benzyl protecting group R7 by catalytic hydrogenation with a palladium catalyst such as palladium on carbon or Pearlman's catalyst under a hydrogen atmosphere provides C8.

The production of compounds of Formula I is depicted in Chart D. Reaction of D1, where X can be but is not limited to I, Br, Cl, OR7, and triflate according to Chart A and B, with an iodinating reagent such as iodine in a solvent such as DMF (Bocchi, V.; Synthesis; 1982; 1096) provides the corresponding iodide D2. Reaction of D2 with 2-propyn-1-ol under Sonogashira coupling conditions (Sakamoto, T.; Chem. Pharm. Bull.; 36; 1988; 2248-52) furnishes D3. Catalytic hydrogenation in the presence of a transition metal such as palladium (Yagi, S.; J. Chem. Soc. Perkin. Trans. 1; 6; 2000; 925-32) or platinum oxide (Gray, G. W.; Mol. Cryst. Liq. Cryst.; 204; 1991; 43-64) affords-D4. Conversion of the terminal alcohol moiety of D4 to a suitable leaving group such as mesylate or halide followed by nucleophilic displacement with an amine derivative such as benzyl amine in a polar solvent such as CH₃CN or DMF furnishes the corresponding amine D5. Reaction of D5 with a palladium catalyst such as Pd(PPh₃)₄ in the presence of a base such as sodium tert-butoxide and/or potassium carbonate in a high boiling solvent such as toluene at elevated temperatures furnishes D6 (Buchwald, S. L.; Tetrahedron; 52; 21; 1996; 7525-7546). Deprotection of the tert-butylcarbamate under acidic conditions such as trifluoroacetic acid in a solvent such as dichloromethane provides D7. Reduction of indole derivatives D7 by a reducing agent such as sodium cyanoborohydride in the presence of an acid such as TFA or acetic acid affords the corresponding indolines D8. Removal of the N-benzyl protecting group by catalytic hydrogenation with a transition metal such as palladium on carbon or Pearlman's catalyst under a hydrogen atmosphere provides D9.

The production of compounds of Formula I is also depicted in Chart E. Compound E1 (according to Chart D) reacts with an oxidant such as the pyridinium dichromate (PDC) in appropriate solvent such DMF affording the carboxylic acid E2 (for additional oxidation procedures see above reference Larock, R. C., Comprehensive Organic Transformation). Treatment of the carboxylic acid with sulfonyl chloride or oxalylchloride in an appropriate solvent such as dichloromethane converts the acid to the corresponding acid chloride E3. Treatment of the acid chloride E3 with a Lewis acid such as aluminum trichloride in an appropriate solvent such as dichloroethane results in intramolecular cyclization to produce E4 (see Osborne, S.; Tet. Lett.; 39; 1988; 8729-32). Deprotection of the tert-butylcarbamate with TFA followed by ketone reduction with a reducing reagent such as triethylsilane in an appropriate solvent such as TFA provides E5 (Taylor, E. W.; Synth. Comm.; 19; 1989; 369). Reduction of indole derivatives E5 by a reducing agent such as sodium cyanoborohydride in the presence of an acid such as TFA or acetic acid (see above Indoles; Sundberg, R. J.; 1996) produces E6. For compounds of Formula I where Y is —CH₂—, c=0 or c=1, and b=2, ring expansion of the cyclohexanone derivative E4 with (trimethylsilyl)diazomethane in the presence of a Lewis acid such as triethyloxonium tetrafluoroborate followed by desilylation with aqueous base affords cycloheptanones E7 (see above reference from Osborne, S.). Reduction of the ketone moiety employing a modified Wolf Kishner procedure with hydrazine hydrate in a solvent such as diethylene glycol followed by treatment with a base such as potassium hydroxide provides E8 (D'Angelo, J.; J. Org. Chem.; 62; 12; 1997; 3890-3901 or see Szmuszkovicz, J.; J. Org. Chem.; 29; 1964; 843-854. For additional reduction procedures see above reference Larock, R. C., Comprehensive Organic Transformation). Reduction of the indole derivatives E8 with a reducing agent such as sodium cyanoborohydride in the presence of an acid such as TFA or acetic acid affords E9.

The production of compounds of Formula I is also depicted in Chart F. De-methylation of F1 (according to Chart A and B) by trimethylsilyl chloride and m-chloroperbenzoic acid (Lavanish, Tet. Lett.; 1973; 3847) provides F2. Alkylation of thiols F2 with either 2-bromoacetic acid or 3-bromoproprionic acid in the presence of a base such as potassium hydroxide and solvent such as acetonitrile (Horaguchi, T.; J. Heterocycl. Chem.; 29; 2; 1992; 503) affords F3 (b=1 to 2). Intramolecular Friedel-Crafts acylation in a single step with a strong acid such as sulfuric acid or polyphosphoric acid in a solvent such as dichloromethane (see above Horaguchi, T. reference or Schultze, H.; Chem. Ber.; 56; 1923; 1819) or in a two step procedure with activation of the acid as the acid chloride followed by Friedel-Crafts acylation (Mann, F. G.; J. Chem. Soc.; 1945; 893 and Cagniant P.; Bull. Soc. Chim. Fr.; 1966; 3674) produces F4 (b=1 or 2). Reduction of the ketone moiety in a two step procedure by initial reduction to an alcohol with sodium borohydride in an alcoholic solvent followed by treatment with triethylsilane and TFA in a non polar solvent such as dichloromethane affords F5 (b=1 or 2) (Chung, J. Y. L.; Tet. Lett.; 33; 33; 1992; 4717). Reduction of the indole derivatives F5 by a reducing agent such as sodium cyanoborohydride in the presence of an acid such as TFA or acetic acid affords F6 (b=1 to 2). For alternative synthetic approaches to the production of compounds of Formula I see above Chung, J. Y. L. reference or Akita, H.; Tet. Lett.; 38; 10; 1997; 1805.

The production of compounds of Formula I is depicted in Chart G. Reaction of G1 (according to Chart C and D) with 48% hydrobromic acid or boron tribromide in a solvent such as dichloromethane furnishes the phenol derivatives G2 (b=1 to 2). Protection of the azepino nitrogen as the tert-butylcarbamate provides G3 (b=1 to 2). Reaction under Mitsunobu conditions with diethylazodicarboxylate and triphenylphosphine in a solvent such as THF (King, J. D.; Tet. Lett.; 32; 52; 1991; 7759-62 and Macor, J. E.; Tet. Lett.; 32; 28; 1991; 3345-48) followed by deprotection of the tert-butylcarbamate with TFA affords G4 derivatives (b=1 to 2). Reduction of the indole derivatives G4 by a reducing agent such as sodium cyanoborohydride in the presence of an acid such as TFA or acetic acid affords G5 (b=1 to 2).

Chart H illustrates an alternative preparation of a subset of compounds of Formula I where c=0 or c=1, and where either R₁ or R₃ is a substituted aryl or heteroaryl substituent. Reaction of H1 with a halogen source such as N-bromosuccinimide, bromine, or N-chlorosuccinimide in a solvent such as DMF or dichloromethane provides regioisomers H2 and H3. Reaction of H2 and H3 under various catalytic transition metal coupling procedures and in the presence of, but not limited to, arylboronic acids and esters, arylorganostannanes, or arylsilanes provides H4 and H5 derivatives. These coupling procedures include, but are not limited to, the Suzuki coupling (for a review see Snieckus, V.; Chem. Rev.; 90; 897; 1990 or for the use of arylchlorides as substrates see Fu, G. C.; Angew. Chem. Int. Ed.; 37; 24; 1998; 3387-88), the Stille coupling (for a review see Beletzkaya, I. P.; J. Organomet. Chem.; 250; 551; 1983), the Heck coupling (for a review see de Meijere, A. and Meyer, F. E.; Angew. Chem. Int. Ed. Engl.; 33; 2379; 1994), and heteroatom carbon coupling reactions (for a review see Buchwald, S. L. and Yang, B. H.; J. Organomet. Chem.; 576; 125; 1999). Deprotection of the tert-butylcarbamate with TFA in a solvent such as dichloromethane affords H6 and H7 derivatives. Reaction of H6 and H7 with an oxidant such as 2,3-dichloro-5,6-dicyano-1,4-benzoquinone (DDQ) or manganese dioxide in a solvent such as THF or dioxane (see above Indoles; Sundberg, R. J.; 1996) provides the indole derivatives H8 and H9. This sequence can be carried out with non-critical variations utilizing parallel synthetic techniques and employing polymer supported reagents as reactants, scavengers, and capture-release reagents to produce libraries of compounds.

The preparation of compounds of Formula I where R₄ is not equal to hydrogen is depicted in Chart I. Deprotection of I1 (according to Charts C to H) with TFA in a solvent such as dichloromethane provides I2. Alkylation of the azepino ring nitrogen in the presence of a base such as potassium carbonate and in a solvent such as acetonitrile affords amine derivatives I3.

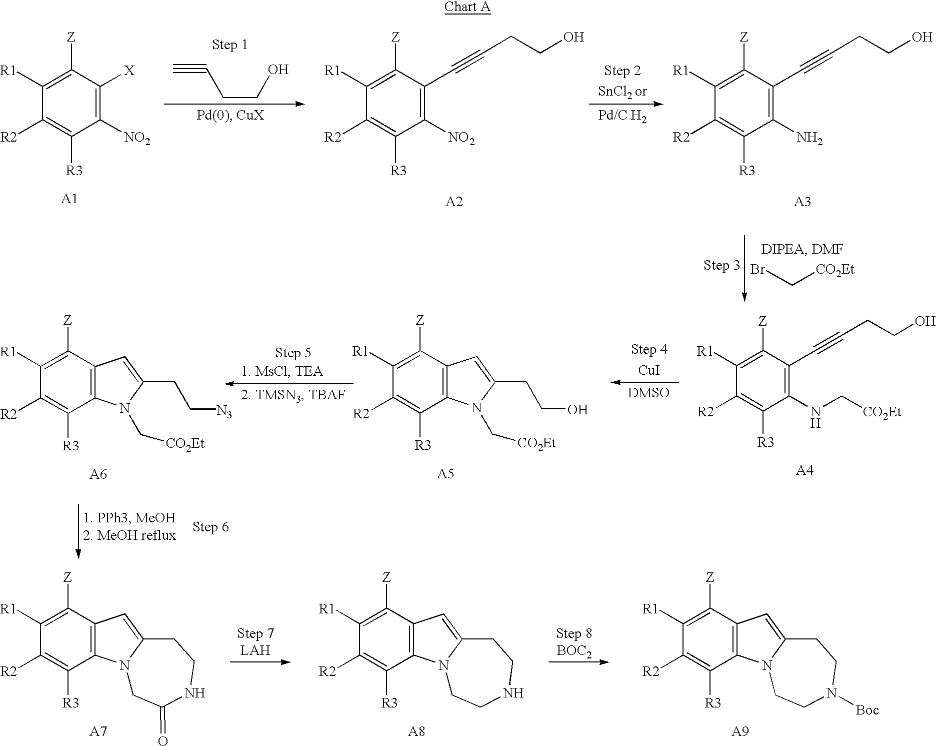

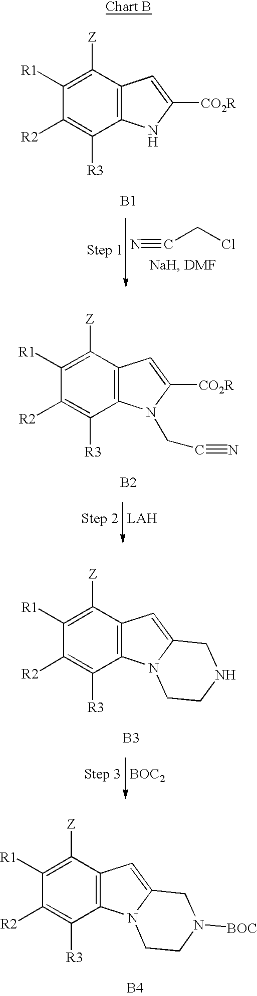

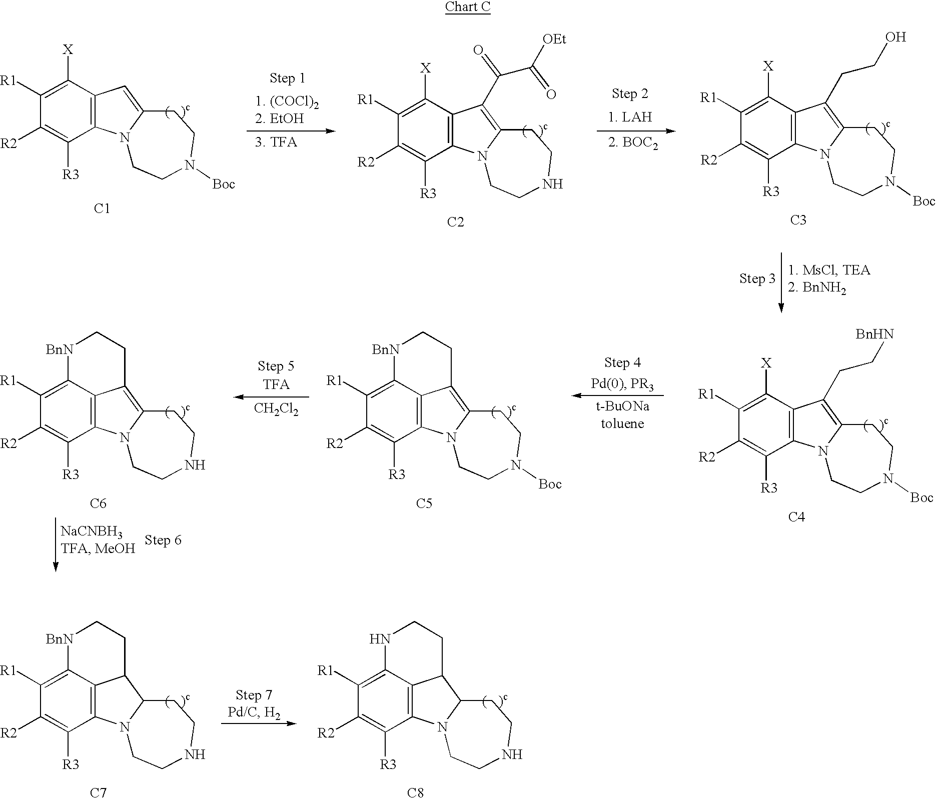

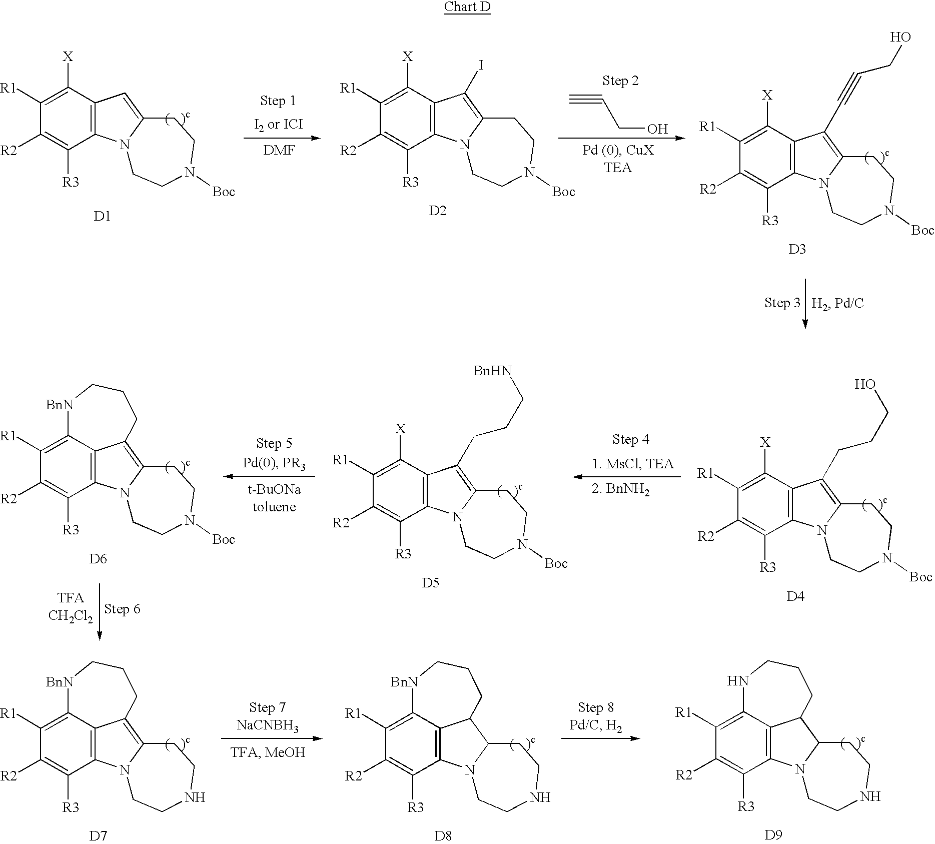

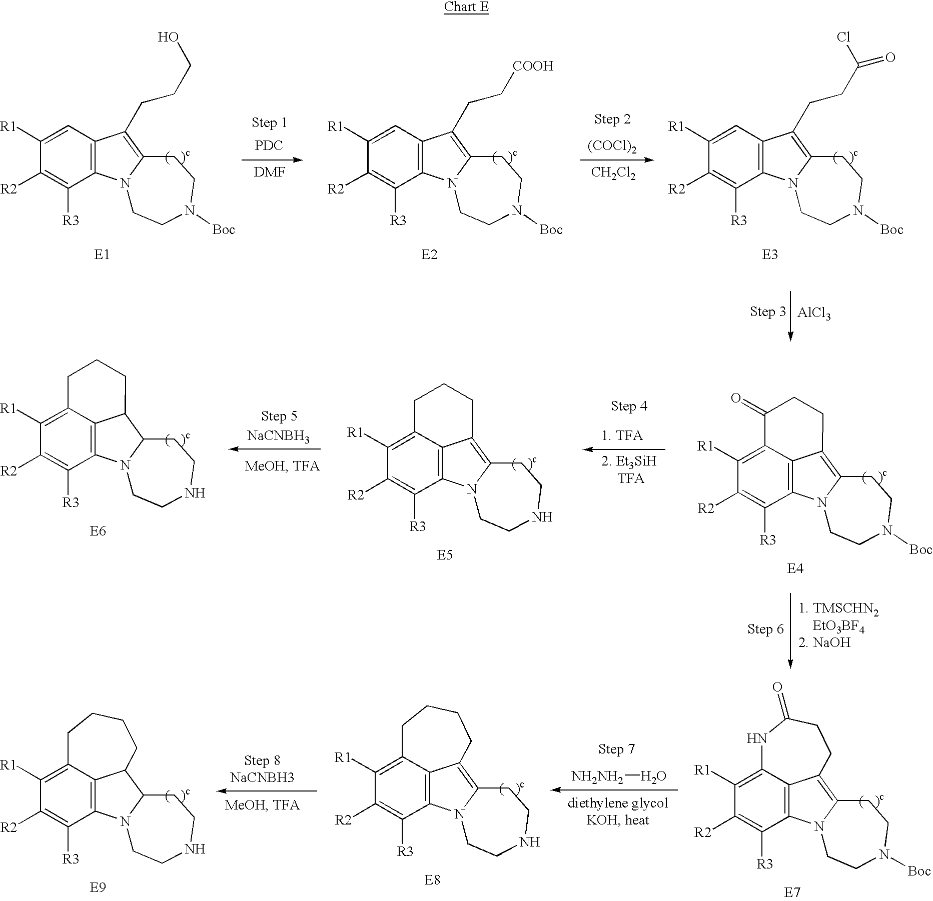

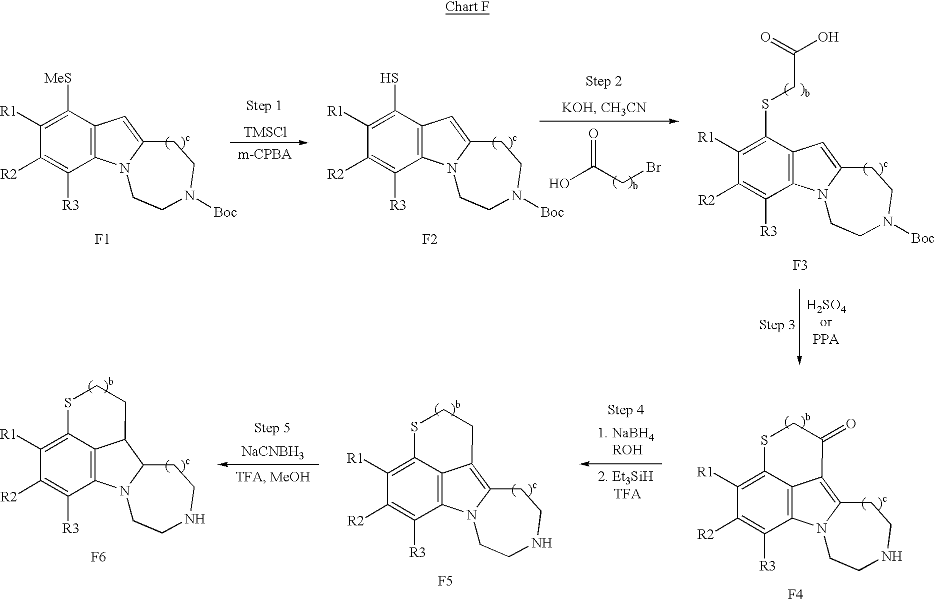

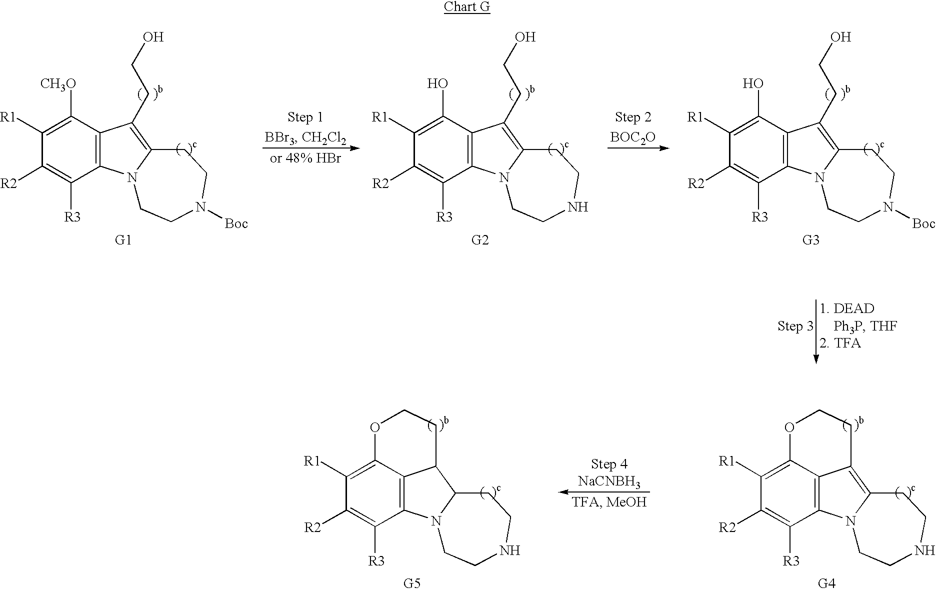

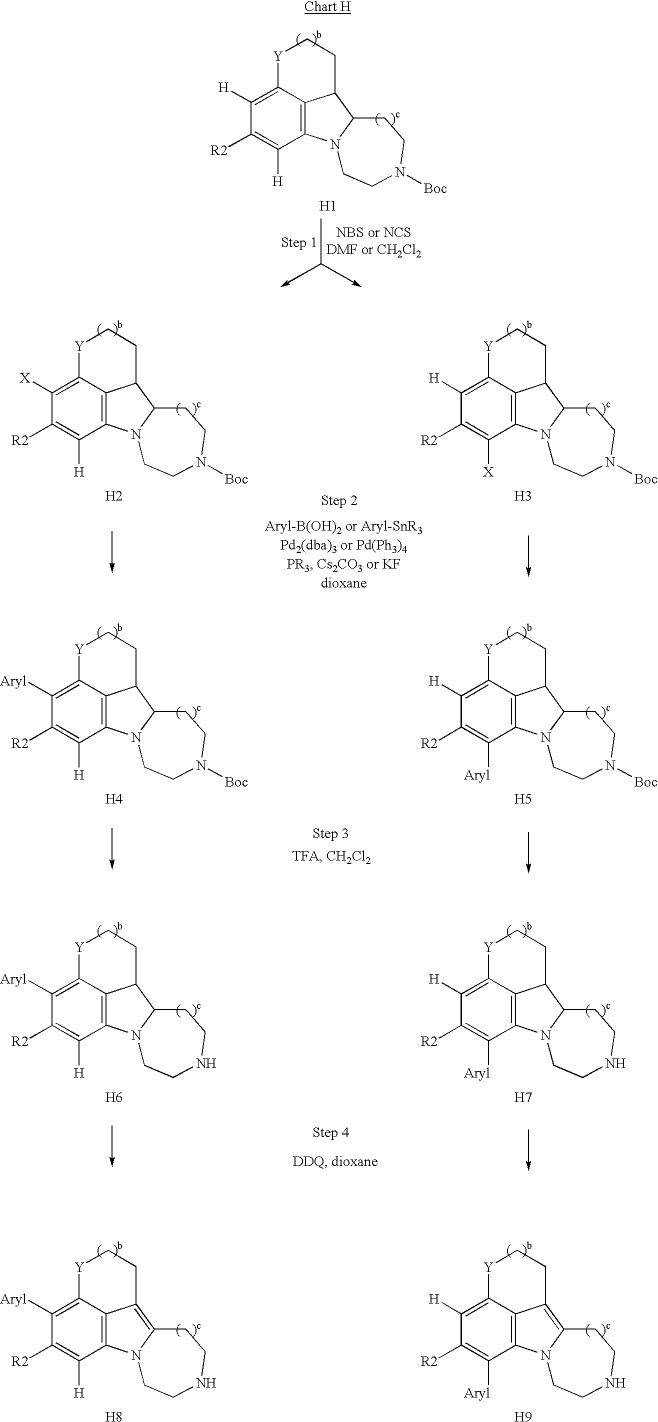

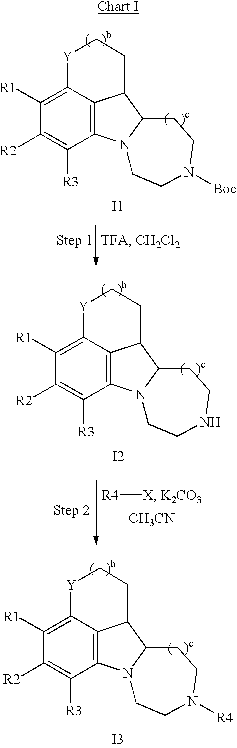

The present invention includes, for example, the following named compounds and their accompanying structural formulas:

6-(2-ethoxyphenyl)-1,2,3,8,9,10,11,12,12a,12b-decahydrobenzocd][1,4]diazepino[1,7-a]indole

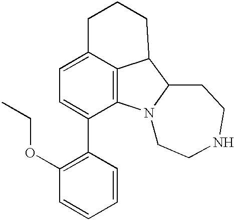

6-(2-ethoxyphenyl)-1,8,9,10,11,12,12a,12b-octahydro-2H-[1,4]diazepino[1,7-a]pyrano[4,3,2-cd]indole

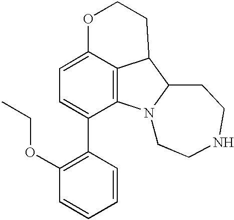

6-(2-ethoxyphenyl)-1,8,9,10,11,12,12a,12b-octahydro-2H-[1,4]diazepino[1,7-a]thiopyrano[4,3,2-cd]indole

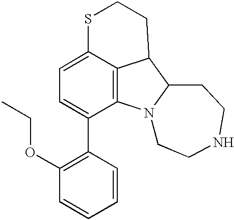

6-(2-ethoxyphenyl)-1,2,3,8,9,10,11,12,12a,12b-decahydro[1,4]diazepino[7′,1′:5,1]pyrrolo[4,3,2-de]quinoline

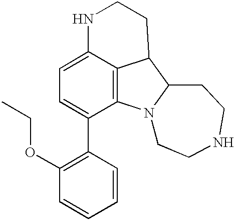

7-(2-ethoxyphenyl)-2,3,4,9,10,11,12,13,13a,13b-decahydro-1H-cyclohepta[cd][1,4]diazepino[1,7-a]indole

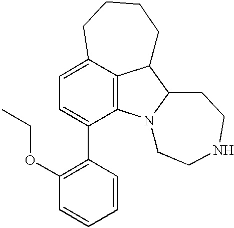

7-(2-ethoxyphenyl)-1,2,3,9,10,11,12,13,13a,13b-decahydro[1,4]diazepino[1,7-a]oxepino[4,3,2-cd]indole

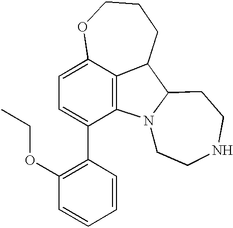

7-(2-ethoxyphenyl)-1,2,3,9,10,11,12,13,13a,13b-decahydro[1,4]diazepino[1,7-a]thiepino[4,3,2-cd]indole

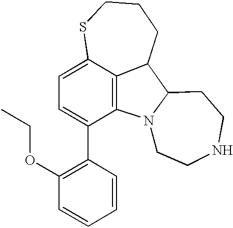

7-(2-ethoxyphenyl)-2,3,4,9,10,11,12,13,13a,13b-decahydro-1H-azepino[4,3,2-cd][1,4]diazepino[1,7-a]indole

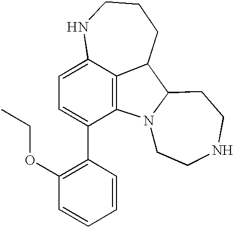

6-(2-ethoxyphenyl)-2,3,8,9,10,11,11a,11b-octahydro-1H-benzo[cd]pyrazino[1,2-a]indole

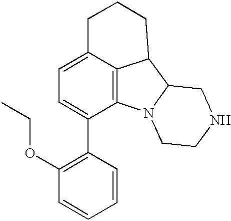

6-(2-ethoxyphenyl)-1,2,8,9,10,11,11a,11b-octahydropyrano[4,3,2-cd]pyrazino[1,2-a]indole

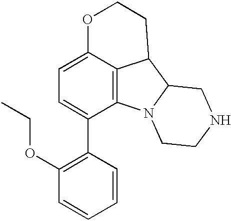

6-(2-ethoxyphenyl)-1,2,8,9,10,11,11a,11b-octahydropyrazino[1,2-a]thiopyrano[4,3,2-cd]indole

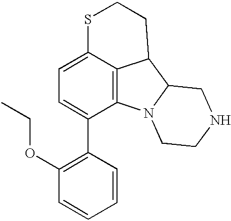

6-(2-ethoxyphenyl)-2,3,8,9,10,11,11a,11b-octahydro-1H-pyrazino[2′,1′:5,1]pyrrolo[4,3,2-de]quinoline

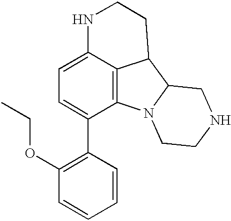

7-(2-ethoxyphenyl)-1,2,3,4,9,10,11,12, 12a, 12b-decahydrocyclohepta[cd]pyrazino[1,2-a] indole

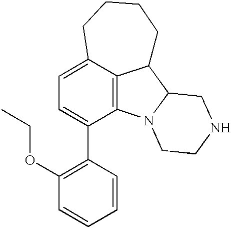

7-(2-ethoxyphenyl)-2,3,9,10,11,12, 12a, 12b-octahydro-1H-oxepino[4,3,2-cd]pyrazino[1,2-a]indole

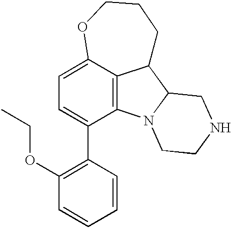

7-(2-ethoxyphenyl)-2,3,9,10,11,12,12a,12b-octahydro-1H-pyrazino[1,2-a]thiepino[4,3,2-cd]indole

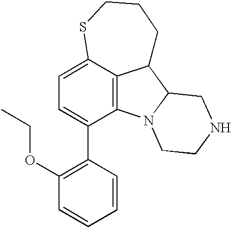

7-(2-ethoxyphenyl)-1,2,3,4,9,10,11,12,12a,12b-decahydroazepino[4,3,2-cd]pyrazino[1,2-a]indole

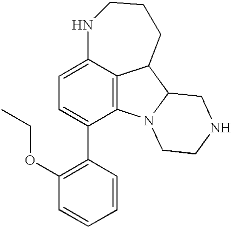

7-(2-ethoxyphenyl)-11-methyl-1,2,3,9,10,11,12,13,13a,13b-decahydro[1,4]diazepino[1,7-a]thiepino[4,3,2-cd]indole

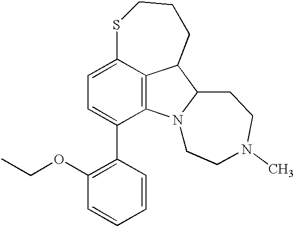

11-cyclopropyl-7-(2-ethoxyphenyl)-1,2,3,9,10,11,12,13,13a,13b-decahydro[1,4]diazepino[1,7-a]thiepino[4,3,2-cd]indole

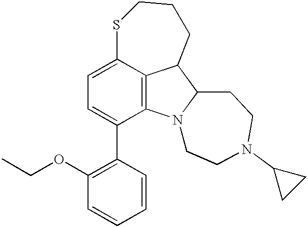

11-allyl-7-(2-ethoxyphenyl)-1,2,3,9,10,11,12,13,13a,13b-decahydro[1,4]diazepino[1,7-a]thiepino[4,3,2-cd]indole

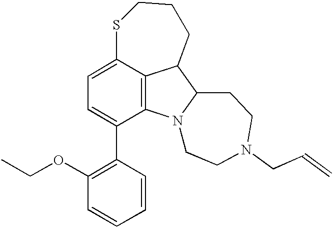

11-benzyl-7-(2-ethoxyphenyl)-1,2,3,9,10,11,12,13,13a,13b-decahydro[1,4]diazepino[1,7-a]thiepino[4,3,2-cd]indole

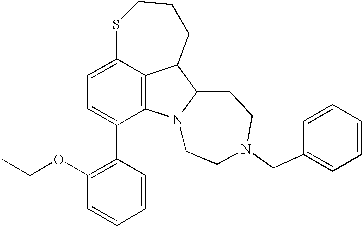

The above named compounds can be prepared by reduction of the corresponding unsaturated compound corresponding to Formula III and as illustrated herein.

In cases where compounds are sufficiently basic or acidic to form stable nontoxic acid or base salts, administration of the compounds as salts may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

Compounds of the present invention can conveniently be administered in a pharmaceutical composition containing the compound in combination with a suitable excipient. Such pharmaceutical compositions can be prepared by methods and contain excipients which are well known in the art. A generally recognized compendium of such methods and ingredients is Remington's Pharmaceutical Sciences by E. W. Martin (Mark Publ. Co., 15th Ed., 1975). The compounds and compositions of the present invention can be administered parenterally (for example, by intravenous, intraperitoneal or intramuscular injection), topically, orally, or rectally.

For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The compounds or compositions can also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

Pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions can be prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The compound is conveniently administered in unit dosage form; for example, containing about 0.05 mg to about 500 mg, conveniently about 0.1 mg to about 250 mg, most conveniently, about 1 mg to about 150 mg of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The compositions can conveniently be administered orally, sublingually, transdermally, or parenterally at dose levels of about 0.01 to about 150 mg/kg, preferably about 0.1 to about 50 mg/kg, and more preferably about 0.1 to about 10 mg/kg of mammal body weight

For parenteral administration the compounds are presented in aqueous solution in a concentration of from about 0.1 to about 10%, more preferably about 0.1 to about 7%. The solution may contain other ingredients, such as emulsifiers, antioxidants or buffers

The exact regimen for administration of the compounds and compositions disclosed herein will necessarily be dependent upon the needs of the individual subject being treated, the type of treatment and, of course, the judgment of the attending practitioner.

The ability of a compound of the invention to act as a 5-HT receptor agonist or antagonist can also be determined using in vitro and in vivo assays that are known in the art. The invention provides compounds of formula I that act as either agonists or as antagonists of one or more 5-HT receptor subtypes. The compounds exemplified herein are 5-HT ligands, which typically displace >50% of a radio-labeled test ligand from one or more 5-HT receptor subtype at a concentration of, for example, about 1 micromolar (μM). The procedures used for testing such displacement are well known and would be readily available to one skilled in the art. See for example, L. W. Fitzgerald et al., Mol. Pharmacol, 2000, 57, 1, 75-81; and D. B. Wainscott, et al., J. Pharmacol Exp Ther, 1996, 276, 2, 720-727.

The invention has been described with reference to various specific and preferred embodiments and techniques. However, it should be understood that many variations and modifications may be made while remaining within the spirit and scope of the invention.

What is claimed is:

1. A compound of formula I: 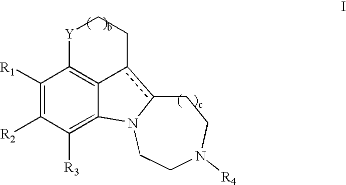
wherein:
b is 1 or 2
c is 0 or 1;
Y is oxy (—O—), thio (—S—), carbonyl (—C(═O)—), NR₅, or CH₂;
the bond represented by ----- is absent or present;
R₁, R₂, and R₃ are each independently hydrogen, halo, —CF₃, —OCF₃, —CN, —NO₂, C₁₋₈alkyl, —OR₆, —SR₆, aryl, (aryl)C₁₋₈alkylene, heteroaryl, (heteroaryl)C₁₋₈alkylene or C₃₋₈ cycloalkyl;
R₄ and R₅ are independently hydrogen, C₁₋₈alkyl, C₃₋₈cycloalkyl, —C(O)O—R₇, —C(O)R₇, —SO₂R₇ or (aryl) C₁₋₈alkylene-;
each R₆ and R₇ is independently hydrogen, C₁₋₈alkyl, C₃₋₈cycloalkyl, aryl or (aryl)C₁₋₈alkylene-;
wherein any aryl or heteroaryl group of R₁, R₂, R₃, R₄, and R₅ is optionally substituted with one or more substituents independently selected from halo, —OH, —CN, —NO₂, —OR₇, —CF₃, —OCF₃, —S(O)₀₋₂C₁₋₈alkyl, C₁₋₈alkyl, phenyl, C₁₋₈alkanoyl, —C(═O)OR₆, —N(R₆)C(═O)NR₆R₇, —N(R₆)C(═O)OR₆, —NR_{c}R_{d}, —C(═O)NR_{c}R_{d}, and —SO₂NR_{c}R_{d};
wherein any C₁₋₈alkyl, or C₁₋₈alkylene group of R₁, R₂, R₃, R₄, and R₅ is optionally substituted with one or more substituents independently selected from oxo (═O), halo, —OH, —CN, —NO₂, —CF₃, —OCF₃, —S(O)₀₋₂C₁₋₈alkyl, C₁₋₈alkyl, C₁₋₈alkoxy, phenyl, C₁₋₈alkanoyl, —C(═O)OR₆, —N(R₆)C(═O)NR₆R₇, —N(R₆)C(═O)OR₆, —NR_{c}R_{d}, —C(═O)NR_{c}R_{d} or —SO₂NR_{c}R_{d};
wherein each R_{c} and R_{d} is independently hydrogen, C₁₋₈alkyl, C₁₋₈alkanoyl, C₁₋₈alkoxycarbonyl, aryl, (aryl)C₁₋₈alkylene-, arylcarbonyl, or aryloxycarbonyl; or R_{c} and R_{d} together with the nitrogen to which they are attached form a pyrrolidino, piperidino, morpholino, or thiomorpholino ring;
wherein any C₁₋₈alkyl, C₁₋₈alkoxy, C₁₋₈alkanoyl, C₁₋₈alkoxycarbonyl, C₁₋₈alkanoyloxy, C₁₋₈alkylene, or C₃₋₈cycloalkyl, of R₁, R₂, R₃, R₄, R₅, R₆, and R₇ is optionally partially unsaturated;
or a pharmaceutically acceptable salt thereof.

2. The compound of claim 1, wherein the bond represented by --- is absent.

3. The compound of claim 1, wherein the bond represented by --- is present.

4. The compound of claim 1, wherein b is 1 and c is 1.

5. The compound of claim 1, wherein b is 1 and c is 0.

6. The compound of claim 1, wherein b is 2 and c is 1.

7. The compound of claim 1, wherein b is 2 and c is 0.

8. The compound of claim 1, wherein Y is oxy.

9. The compound of claim 1, wherein Y is thio.

10. The compound of claim 1, wherein Y is carbonyl.

11. The compound of claim 1, wherein Y is —NR₅—.

12. The compound of claim 1, wherein Y is —CH₂—.

13. The compound of claim 1, wherein R₁, R₂ and R₃ are independently hydrogen, halo, C₁₋₈alkyl, —OR₆, or —SR₆.

14. The compound of claim 13, wherein R₁, R₂ and R₃ are independently hydrogen, halo or C₁₋₈alkyl.

15. The compound of claim 14, wherein R₁, R₂ and R₃ are independently hydrogen or halo.

16. The compound of claim 14, wherein R₁, R₂ and R₃ are independently hydrogen or C₁₋₈alkyl.

17. The compound of claim 1, wherein R₁, R₂ and R₃ are independently hydrogen, aryl or heteroaryl.

18. The compound of claim 17, wherein R₁, is aryl, substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

19. The compound of claim 18, wherein R₁, is phenyl, optionally substituted with one or more fluoro, chloro, bromo, —OH, —CN, —NO₂, —CH₃, —CF₃, —OCF₃, methoxy, ethoxy, propoxy, or isopropoxy.

20. The compound of claim 19, wherein R₁ is phenyl substituted at the 2-position with one or more fluoro, chloro, bromo, —OH, —CN, —NO₂, —CH₃, —CF₃, —OCF₃, methoxy, ethoxy, propoxy, or isopropoxy.

21. The compound of claim 20, wherein R₁ is 2-ethoxyphenyl, 2-trifluoro-phenyl, 2-chlorophenyl or 2-methylphenyl.

22. The compound of claim 17, wherein R₁ is heteroaryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

23. The compound of claim 22, wherein R₁ is heteroaryl substituted with one or more fluoro, chloro, bromo, —OH, —CN, —NO₂, —CF₃, —OCF₃, C₁₋₈alkyl, or C₁₋₈alkoxy.

24. The compound of claim 17, wherein R₂ is aryl substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

25. The compound of claim 24, wherein R₂ is phenyl, optionally substituted with one or more fluoro, chloro, bromo, —OH, —CN, —NO₂, —CH₃, —CF₃, —OCF₃, methoxy, ethoxy, propoxy, or isopropoxy.

26. The compound of claim 25, wherein R₂ is 2-ethoxyphenyl, 2-trifluoro-phenyl, 2-chlorophenyl or 2-methylphenyl.

27. The compound of claim 17, wherein R₂ is heteroaryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

28. The compound of claim 27, wherein R₂ is heteroaryl substituted with one or more fluoro, chloro, bromo, —OH, —CN, —NO₂, —CF₃, —OCF₃, C₁₋₈alkyl, or C₁₋₈alkoxy.

29. The compound of claim 17, wherein R₃ is aryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

30. The compound of claim 29, wherein R₃ is phenyl, optionally substituted with one or more fluoro, chloro, bromo, —OH, —CN, —NO₂, —CH₃, —CF₃, —OCF₃, methoxy, ethoxy, propoxy, or isopropoxy.

31. The compound of claim 30, wherein R₃ phenyl substituted at the 2-position with one or more fluoro, chloro, bromo, —OH, —CN, —NO₂, —CH₃, —CF₃, —OCF₃, methoxy, ethoxy, propoxy, or isopropoxy.

32. The compound of claim 31, wherein R₃ is 2-ethoxyphenyl, 2-trifluoro-phenyl, 2-chlorophenyl or 2-methylphenyl.

33. The compound of claim 17, wherein R₃ is heteroaryl, optionally substituted with one or more halo, —OH, —CN, —NO₂, —CF₃, —OCF₃, C₁₋₈alkyl, C₁₋₈alkoxy, phenyl, NR_{c}R_{d}, or —C(═O)NR_{c}R_{d}.

34. The compound of claim 33, wherein R₃ is heteroaryl substituted with one or more fluoro, chloro, bromo, —OH, —CN, —NO₂, —CF₃, —OCF₃, C₁₋₈alkyl, or C₁₋₈alkoxy.

35. The compound of claim 1, wherein R₄ is hydrogen.

36. The compound of claim 1, wherein R₄ is C₁₋₈ alkyl, C₃₋₈ cycloalkyl or (aryl) C₁₋₈ alkylene.

37. The compound of claim 1, wherein R₄ is a nitrogen protecting group.

38. The compound of claim 37, wherein R₄ is —C(O)O-t-Bu, —C(O)CF₃, —(O)OCH₂Ph, or —CH₂—Ph.

39. The compound of claim 1, wherein R₅ is a nitrogen protecting group.

40. The compound of claim 39, wherein R₅ is —C(O)O-t-Bu, —C(O)CF₃, —(O)OCH₂Ph, or —CH₂—Ph.

41. The compound of claim 1, wherein the compound is: 6-(2-ethoxyphenyl)-1,2,3,8,9,10,11,12,12a,12b-decahydrobenzocd][1,4]diazepino[1,7-a]indole; 6-(2-ethoxyphenyl)-1,8,9,10,11,12,12a,12b-octahydro-2H-[1,4]diazepino[1,7-a]pyrano[4,3,2-cd]indole; 6-(2-ethoxyphenyl)-1,8,9,10,11,12,12a,12b-octahydro-2H-[1,4]diazepino[1,7-a]thiopyrano[4,3,2-cd]indole; 6-(2-ethoxyphenyl)-1,2,3,8,9,10,11,12,12a,12b-decahydro[1,4]diazepino[7′,1′:5,1]pyrrolo[4,3,2-de]quinoline; 7-(2-ethoxyphenyl)-2,3,4,9,10,11,12,13,13a,13b-decahydro-1H-cyclohepta[cd][1,4]diazepino[1,7-a]indole; 7-(2-ethoxyphenyl)-1,2,3,9,10,11,12,13,13a,13b-decahydro[1,4]diazepino[1,7-a]oxepino[4,3,2-cd]indole; 7-(2-ethoxyphenyl)-1,2,3,9,10,11,12,13,13a,13b-decahydro[1,4]diazepino[1,7-a]thiepino[4,3,2-cd]indole; 7-(2-ethoxyphenyl)-2,3,4,9,10,11,12,13,13a,13b-decahydro-1H-azepino[4,3,2-cd][1,4]diazepino[1,7-a]indole; 6-(2-ethoxyphenyl)-2,3,8,9,10,11,11a,11b-octahydro-1H-benzo[cd]pyrazino[1,2-a]indole; 6-(2-ethoxyphenyl)-1,2,8,9,10,11,11a,11b-octahydropyrano[4,3,2-cd]pyrazino[1,2-a]indole; 6-(2-ethoxyphenyl)-1,2,8,9,10,11,11a,11b-octahydropyrazino[1,2-a]thiopyrano[4,3,2-cd]indole; 6-(2-ethoxyphenyl)-2,3,8,9,10,11,11a,11b-octahydro-1H-pyrazino[2′,1′:5,1]pyrrolo[4,3,2-de]quinoline; 7-(2-ethoxyphenyl)-1,2,3,4,9,10,11,12,12a,12b-decahydrocyclohepta[cd]pyrazino[1,2-a]indole; 7-(2-ethoxyphenyl)-2,3,9,10,11,12,12a,12b-octahydro-1H-oxepino[4,3,2-cd]pyrazino[1,2-a]indole; 7-(2-ethoxyphenyl)-2,3,9,10,11,12,12a,12b-octahydro-1H-pyrazino[1,2-a]thiepino[4,3,2-cd]indole; 7-(2-ethoxyphenyl)-1,2,3,4,9,10,11,12,12a,12b-decahydroazepino[4,3,2-cd]pyrazino[1,2-a]indole.

42. The compound of claim 36, wherein the compound is 7-(2-ethoxyphenyl)-11-methyl-1,2,3,9,10,11,12,13,13a,13b-decahydro[1,4]diazepino[1,7-a]thiepino[4,3,2-cd]indole; 11-cyclopropyl-7-(2-ethoxyphenyl)-1,2,3,9,10,11,12,13,13a,13b-decahydro[1,4]diazepino[1,7-a]thiepino[4,3,2-cd]indole; 11-allyl-7-(2-ethoxyphenyl)-1,2,3,9,10,11,12,13,13a,13b-decahydro[1,4]diazepino[1,7-a]thiepino[4,3,2-cd]indole; or 11-benzyl-7-(2-ethoxyphenyl)-1,2,3,9,10,11,12,13,13a,13b-decahydro[1,4]diazepino[1,7-a]thiepino[4,3,2-cd]indole.

43. A pharmaceutical composition comprising a compound of claim 1 and a pharmaceutically acceptable excipient.

44. A compound of claim 1 for use in medical diagnosis or therapy.

45. The compound of claim 44, wherein the therapy is the treatment of a disease or disorder of the central nervous system.

46. The use of a compound of claim 1 to prepare a medicament for treating or preventing a disease or disorder of the central nervous system.

47. A method for treating a disease or condition in a mammal in need thereof, wherein the 5-HT receptor is implicated and modulation of 5-HT function is desired comprising administering a therapeutically effective amount of a compound of claim 1 to the mammal.

48. A method for treating or preventing a disease or disorder of the central nervous system in a mammal in need thereof comprising administering a therapeutically effective amount of a compound of claim 1 to the mammal.
